SEQUENCE LISTING (6 sequences)

SEQ ID NO: 1 - DNA, 25 bp, Artificial Sequence
  Description of Artificial Sequence  Synthetic primer
ccgccttgagcggcaggtttaaagg

SEQ ID NO: 2 - DNA, 24 bp, Artificial Sequence
  Description of Artificial Sequence  Synthetic primer
catgtcaccaagctcgacgaggtg

SEQ ID NO: 3 - DNA, 25 bp, Artificial Sequence
  Description of Artificial Sequence  Synthetic primer
ccgccttgagcggcaggtttaaagg

SEQ ID NO: 4 - DNA, 20 bp, Artificial Sequence
  Description of Artificial Sequence  Synthetic primer
tggatgtggaatgtgtgcga

SEQ ID NO: 5 - protein, 22 aa, Artificial Sequence
  Description of Artificial Sequence  Synthetic peptide
MTYAANILPHHPARGTFEDFTC

SEQ ID NO: 6 - protein, 12 aa, Artificial Sequence
  Description of Artificial Sequence  Synthetic peptide
GTCNAVTMDSDF

(19) United States
(12) Patent Application Publication
(10) Pub. No.: US 2007/0197583 A1
(43) Pub. Date: Aug. 23, 2007

(54) Novel compositions and methods for modulation of the acid-sensing ion channel (ASIC) for the treatment of anxiety and drug addiction

(76) Inventors: Michael J. Welsh, Riverside, IA (US); John A. Wemmie, Iowa City, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 10/659,467

(22) Filed: Sep. 10, 2003

Related U.S. Application Data
(63) Continuation-in-part of application No. 10/112,280, filed on Mar. 29, 2002, now abandoned.

(51) Int. Cl. A61K 31/47

(52) U.S. Cl. 514/310

(57) ABSTRACT

This invention provides novel compositions and methods for modulating acid-sensing ion channels (ASIC) function comprising ASIC and derivatives thereof; methods for modulating ASIC function and methods for treating anxiety or anxiety disorders and drug addiction using the novel compositions of the invention; and a method for increasing synaptic plasticity are described.

### CROSS-REFERENCE TO RELATED APPLICATION

This continuation-in-part application claims the benefit of priority under 35 U.S.C. § 119, to U.S. application Ser. No. 10/112,280, filed on Mar. 29, 2002, the entire contents of which is incorporated by reference herein.

### FIELD OF THE INVENTION

This invention relates to acid-sensing ion channel (ASIC) agonists, antagonists and modulators. In particular, this invention relates to methods of treatment for anxiety, including but not limited to, generalized anxiety disorders and acute stress reactions, drug addiction and fear conditioning, methods of identifying potential new therapeutic agents and pharmaceutical compositions for treatment of these disorders by assaying compounds which modulate the acid-sensing ion channel (ASIC).

### BACKGROUND OF THE INVENTION

The present invention relates to methods of treatment for CNS disorders which have been attributed to neurotransmitter system dysfunction. CNS disorders are a type of neurological disorder. CNS disorders can be drug induced; can be attributed to genetic predisposition, infection or trauma; or can be of unknown etiology. CNS disorders comprise neuropsychiatric disorders, neurological diseases and mental illnesses; and include neurodegenerative diseases, behavioral disorders, cognitive disorders and cognitive affective disorders. There are several CNS disorders whose clinical manifestations have been attributed to CNS dysfunction (i.e., disorders resulting from inappropriate levels of neurotransmitter release, inappropriate properties of neurotransmitter receptors, and/or inappropriate interaction between neurotransmitters and neurotransmitter receptors). Several CNS disorders can be attributed to a cholinergic deficiency, a dopaminergic deficiency, an adrenergic deficiency and/or a serotonergic deficiency. CNS disorders of relatively common occurrence include presenile dementia (early onset Alzheimer's disease), senile dementia (dementia of the Alzheimer's type, Parkinsonism including Parkinson's disease), Huntington's chorea, tardive dyskinesia, hyperkinesia, mania, attention deficit disorder, anxiety, dyslexia, schizophrenia and Tourette's syndrome.

It has been recognized that rapid acidification of extracellular pH in CNS disorders evokes a transient cation current in central neurons (Groul et al., 1980; Krishtal and Pidoplichko, 1981). As of yet due to the brain pH being tightly regulated in vivo, the physiological significance of this observation has been unclear. It had been hypothesized only that H⁺-gated currents within the brain might be activated during synaptic transmission due to the EPSPs acidifying the extracellular fluid in hippocampal slices (Krishtal et al., 1987). The discovery of acid-sensing ion channels (ASICs), acid-sensing members of the DEG/ENaC family, has presented an opportunity to further explore the unknown physiological role of neuronal H⁺-evoked currents. The present invention discovers how the acid-sensing ion channel (ASIC) contributes to synaptic plasticity in the hippocampal circuit and areas enriched with strong excitatory synaptic input such as the glomerulus of the olfactory bulb, whisker barrel cortex, cingulated cortex, striatum, nucleus accumbens, amygdala, and cerebellar cortex. The present invention thus teaches the previously unknown effect of ASIC disruption on H⁺-evoked currents in the brain thereby providing methods of treatment for anxiety, anxiety disorders, drug addiction and improved synaptic plasticity for fear conditioning all of which have been attributed to CNS disorders which display neurotransmitter system dysfunction.

Conventional methods for treatment of anxiety disorders, drug addiction, and for treatment of fear conditioning have been endogenous substances and drugs that modulate noxious responses by acting on neurons from the CNS. Currently, patients are often given benzodiazepines to relieve anxiety which bind to GABA_{A} receptors and increase Cl⁻ conductance of the GABA_{A} receptors. GABA_{A} receptors are widely distributed in the CNS where benzodiazepines bind to the α subunits thereby facilitating Cl⁻conductance. Earlier treatments have had various side effects which are undesirable, such as tolerance to the drug, dependence and withdrawal symptoms, to name a few. The GABA_{A} receptors are pentamers made up of various combinations of six α, four β, four γ, one δ, and one ε subunit which endows them with considerably different properties from one location to another making it difficult to effectively treat a patients condition due to the varied combination of subunits. The GABAγ₂ subunit is also required for full sensitivity to benzodiazepines therefore often times it is observed in patients without this subunit that there is a decreased sensitivity to the drugs and actually increased anxiety behavior. Therefore there has thus been a long felt need in the art to obtain a treatment, which is void of withdrawal and discontinuation effects and does not cause development of tolerance in patients.

For the foregoing reasons, there is a need for determination, characterization and application of ASIC modulation of synaptic plasticity in the amygdala to provide methods of treatment for anxiety, drug addiction, and fear conditioning.

Accordingly, a primary objective of the invention is methods of treatment for anxiety and anxiety disorders, drug addiction and loss of memory using ASIC antagonists or agonists, respectively.

Another objective of the invention is a method for identifying new therapeutic agents for the treatment of conditions such as anxiety and anxiety disorders, drug addiction, and fear conditioning by screening compounds for their ability to modulate the ASIC channel.

Another objective is to provide pharmaceutical compositions for the treatment of anxiety and anxiety disorders, drug addiction and fear conditioning using ASIC antagonists or agonists, respectively, thereby affecting those regions supporting high levels of synaptic plasticity.

A further objective of the invention is a method to enhance memory and learning, for example, by way of neural mechanisms during fear conditioning, activating ASIC or utilizing new therapeutic agents.

### SUMMARY OF THE INVENTION

The present invention is directed to methods of treatment for anxiety and anxiety disorders, drug addiction and fear conditioning by providing ASIC antagonists and agonists, respectively that are linked to synaptic plasticity in the hippocampal circuit and amygdala. According to the invention, a drug screening protocol for identifying new therapeutic agents based on their ability to act as an ASIC antagonist or agonist thereby providing a treatment for anxiety and anxiety disorders, drug addiction and the neural mechanisms of fear conditioning are presented. In addition, the present invention also relates to new pharmaceutical compositions comprising an ASIC antagonist and a pharmaceutically acceptable carrier to treat anxiety, anxiety disorders and drug addiction and an ASIC agonist and a pharmaceutically acceptable carrier to treat fear conditioning.

Based on this finding, therapeutic agents that can activate or block ASIC will have less severe side effects then currently utilized agents, such as benzodiazepines, and will be better tolerated treatments for neurologic damage that results from anxiety and anxiety disorders, drug addiction and for memory loss by directly targeting the newly discovered ASIC without the addictive effects of the previously prescribed therapeutic agents. Determining how one finds an ASIC antagonist or ASIC agonist is suggested through protein localization utilizing immunohistochemistry. The present invention further identifies the function of acid-gated currents in general and H+-gated DEG/ENaC channels that potentiates the effects of acid-sensing ion channels molecular identity and physiologic function which has remained unknown until now thereby allowing for new methods of treatment of anxiety and anxiety disorders, drug addiction and fear conditioning.

### DEFINITIONS

For purposes of this application the following terms shall have the definitions recited herein. Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUM Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The term “antibody” includes reference to antigen binding forms of antibodies (e.g., Fab, F(ab)₂). The term “antibody” frequently refers to a polypeptide substantially encoded by an immunoglobulin genes, or fragments thereof which specifically bind and recognize an analyte (antigen). However, while various antibody fragments can be defined in terms of the digestion of an intact antibody, one of skill will appreciate that such fragments may be synthesized de novo either chemically or by utilizing recombinant DNA methodology.

Thus, the term antibody, as used herein, also includes antibody fragments such as single chain Fᵥ, chimeric antibodies (i.e., comprising constant and variable regions from different species), humanized antibodies (i.e., comprising a complementarily determining region (CDR) from a non-human source) and heteroconjugate antibodies (e.g., bispecific antibodies).

The term “anxiety” includes without limitation the unpleasant emotion state consisting of psychophysiological responses to anticipation of unreal or imagined danger, ostensibly resulting from unrecognized intrapsychic conflict. Physiological concomitants include increased heart rate, altered respiration rate, sweating, trembling, weakness, and fatigue; psychological concomitants include feelings of impending danger, powerlessness, apprehension, and tension. Dorland's Illustrated Medical Dictionary, W. B. Saunders Co., (2000). Anxiety is often observed to be increased where there is a deficit of ASIC thereby affecting the acquisition and expression of anxiety.

As used herein, “anxiety disorder” includes without limitation mental disorders in which anxiety and avoidance behavior predominate. Dorland's Illustrated Medical Dictionary, W. B. Saunders Co., (2000) and Stedman's Medical Dictionary, Williams & Wilkins, 26th ed. (1995). Examples include without limitation panic attack, agoraphobia, panic disorder, acute stress disorder, chronic stress disorder, specific phobia, simple phobia, social phobia, substance induced anxiety disorder, organic anxiety disorder, obsessive compulsive disorder, post-traumatic stress disorder, generalized anxiety disorder, and anxiety disorder NOS. Other anxiety disorders are characterized in Diagnostic and Statistical Manual of Mental Disorders (American Psychiatric Association 4th ed. 2000).

As used herein the term “ASIC receptor agonist” includes any compound which causes activation of the ASIC receptor. This includes both competitive and non-competitive agonists as well as prodrugs which are metabolized to ASIC agonists upon administration, as well as analogs of such compounds disclosed by the assays enclosed herein to be active ASIC agonists.

As used herein the term “ASIC receptor antagonist” includes any compound which causes inhibition of the ASIC receptor. This includes both competitive and non-competitive antagonists as well as prodrugs which are metabolized to ASIC antagonists upon administration, as well as analogs of such compounds disclosed by the assays enclosed herein to be active ASIC antagonists.

As used herein the term “associative learning” refers to a reflex behavior that is elicited automatically by an environmental stimulus. A stimulus is something in the environment that elicits a response. There are two types of associative learning: (1) Classical conditioning whereby learning occurs with the pairing of stimuli, and (2) Operant conditioning whereby learning occurs when a response made leads to a consequence.

The term “derivative” as used herein refers to a substance produced from another substance either directly or by modification or partial substitution.

As used herein the term “drug addiction” refers to a habitual psychological and physiological dependence on a substance that is beyond voluntary control, where the substance is including but not limited to, alcohol, amphetamine, cocaine, heroin, inhalants, morphine, nicotine, opiates, psychoactive drugs, compulsive disorder, depression, headache, and drug or alcohol related withdrawal symptoms

As used herein the term “fear conditioning” refers to the process of acquiring, developing, educating, establishing, learning, or training responses in a patient having an identifiable stimulus including, but not limited to, apprehension, dread or alarm.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not entirely linear. For instance, polypeptides may be branched as a result of posttranslation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine-containing and the methionine-less amino terminal variants of the protein of the invention.

As used herein, the term “pharmaceutically acceptable carrier” refers to any carrier, diluent, excipient, wetting agent, buffering agent, suspending agent, lubricating agent, adjuvant, vehicle, delivery system, emulsifier, disintegrant, absorbent, preservative, surfactant, colorant, flavorant, or sweetener, preferably non-toxic, that would be suitable for use in a pharmaceutical composition.

As used herein, “pharmaceutically acceptable equivalent” includes, without limitation, pharmaceutically acceptable salts, hydrates, metabolites, prodrugs and isosteres. Many pharmaceutically acceptable equivalents are expected to have the same or similar in vitro or in vivo activity as the compounds of the invention.

As used herein, the terms “pharmaceutically effective” or “therapeutically effective” shall mean an amount of each active component of the pharmaceutical composition (i.e. ASIC1 receptor blocker or activator) or method that is sufficient to show a meaningful patient benefit, i.e., treatment, prevention, amelioration, or a decrease in the frequency of the condition or symptom being treated, to block the effect of the ASIC1 receptor as determined by the methods and protocols disclosed herein. When applied to an individual active ingredient, administered alone, the term refers to that ingredient alone. When applied to a combination, the term refers to combined amounts of the active ingredients that result in the therapeutic effect, whether administered in combination, serially or simultaneously.

As used herein, unless otherwise defined in conjunction with specific diseases or disorders, “treating” refers to: (i) preventing a disease, disorder or condition from occurring in an animal or human that may be predisposed to the disease, disorder and/or condition but has not yet been diagnosed as having it; (ii) inhibiting the disease, disorder or condition, i.e., arresting its development; and/or (iii) relieving the disease, disorder or condition, i.e., causing regression of the disease, disorder and/or condition.

### BRIEF DESCRIPTION OF THE DRAWINGS

FIGS. 1A-1D demonstrate ASIC1 immunolocalization in the forebrain. (A) Coronal sections were stained for Niss1 substance or immunolabeled for ASIC1 protein in +/+ and −/− mice. Areas marked by dashed lines in the Niss1-stained section are the areas dissected to prepare protein extracts for Western blotting in D and FIG. 6B. Asterisks in ASIC1 +/+ hemisphere denote areas of nonspecific staining that did not occur bilaterally or in multiple sections. (B and C) enlarged images of dentate gyrus and CA1 respectively. (D) Western blot of ASIC1 protein in 100 μg protein extract from dentate gyrus and CA1. amg, amygdala; cc, corpus callosum; dg, dentate gyrus; ec, external capsule; ect, ectorhinal cortex; En, endopiriform nuclei; fi, fimbria; Hb, habenula; H, hilus (polymorphic layer); ic, internal capsule; LTh, lateral thalamus; MS, medial septal nuclei; PAC, parietal association cortex; Pir, piriform cortex; PCg, posterior cingulate cortex; PRh, perirhinal cortex; S1BF, somatosensory barrel field; Th thalamus.

FIGS. 2A-2C demonstrates ASIC1 immunolocalization in the cortex. (A and B) Immunolabeling in the posterior (post.) cingulate cortex. Stripes extending through layer ii are labeled with an arrowhead. Positive-staining pyramidal cells in layer III are labeled with arrows. *ASIC1-specific staining in layer I. (C) ASIC1 immunostaining is also elevated in layer III of barrel cortex.

FIG. 3 shows immunolocalization of ASIC1 in the sensorimotor cortex and striatum. Coronal sections through the forebrain were stained for Niss1 substance, hematoxylin and eosin (H&E), or ASIC1 protein in ASIC1 +/+ or −/− mice. Center row, staining of representative coronal slices. Top row, insets of somatosensory cortex at higher magnification. Bottom row, insets of external capsule/corpus callosum and striatum at higher magnification. White matter tracts are labeled with arrows. ASIC1 immunolabeling was noticeably reduced in the white matter tracts. Areas of staining that were not present bilaterally and not present in multiple slices, suggesting nonspecific staining, are marked with an asterisk. Aca, anterior commissure; Acb, accumbens nucleus; cc, corpus callosum; Cg cingulate cortex; CPu, caudate/putamen (striatum); ec, external capsule; M1, primary motor cortex; Pir, piriform cortex; S1, somatosensory cortex; VP, ventral pallidum; Tu, olfactory tubercle.

FIG. 4 demonstrates immunolocalization of ASIC1 in the olfactory bulb. Coronal sections through the olfactory bulb were stained for Niss1 substance or immunolabeled for ASIC1 protein in ASIC1 +/+ and −/− mice. Higher magnifications at bottom demonstrate ASIC1 immunostaining in glomeruli (arrows). E/OV, ependymal and subependymal layer/olfactory ventricle; EPI, external plexiform layer; GI, glomerular layer; Gr, granule cell layer; IPI, internal plexiform layer; Mi, mitral cell layer; ON, olfactory nerve layer.

FIGS. 5A-5C demonstrates immunolocalization of ASIC1 in the cerebellum. (A) shows ASIC1 immunohistochemistry in coronal sections of the cerebellum. (B) shows immunohistochemistry in parasagittal sections of the cerebellum. (C) demonstrates immunostaining with anti-calbindin D-28K antibody in fresh frozen tissue. 4V, fourth ventricle; DN, deep cerebellar nuclei; Gc, granule cell layer; ML, molecular layer; Pc, pyramidal cell layer; WM, white matter.

FIGS. 6A-6B shows ASIC1 immunolocalization in the amygdala complex. (A) ASIC immunolocalization in the amygdala complex. Bla, basolateral nucleus; Ce, central nucleus; La, lateral nucleus. (B) Western blotting of ASIC1 in 100 μg of protein extract per lane isolated from indicated brain region. Cos-7 cells transfected with mASIC1, cos. Due to the entire cerebellum being used to generate the cb extract, the subcortical structures with little ASIC1 may have diluted out the high expression level seen by immunohistological staining in the cerebellar cortex (FIG. 5). +/+ and −/− whole brain extract from ASIC1 +/+ and −/− mouse; amg, amygdala; cb, cerebellum; dg, dentate gyrus; Hb, habenula; H, hilus (polymorphic layer); S1BF, somatosensory barrel field; Th, thalamus; PAC, parietal association cortex; PCg, posterior cingulate cortex.

FIGS. 7A-7C are graphs illustrating proton-gated currents in amygdala neurons. (A and B) demonstrate representative recordings of pH 5 evoked response in amygdala neurons from ASIC1 +/+ and −/− mice. (C) demonstrates average current density of peak pH 5-evoked response in amygdala neurons from ASIC1 +/+ (n=14) and −/− (n=18) mice and hippocampal neurons from ASIC1 +/+ mice (n=67; *p<0.01).

FIGS. 8A-8D are graphs illustrating behavioral analysis of learned fear. (A) shows cured fear conditioning. The amount of freezing in 1 min intervals was determined during training (A). (B) shows cured fear conditioning. The amount of freezing in 1 min intervals was determined during testing (B). During testing, the ASIC1 −/− mice froze significantly less than +/+ controls with the presentation of the conditioned stimulus (intervals 4-9) (p=0.02) (+/+, n=5; −/−, n=9). (C and D) demonstrates the context fear conditioning. The difference in freezing between +/+ and −/−/mice was significant during training (intervals 4-6; p=0.002) and during testing (p=0.03) (+/+, n=7; −/−, n=8). Foot shock, arrows; tone, bars. Statistical significance was tested by ANOVA with repeated measures.

### DETAILED DESCRIPTION OF THE PREFERRED EMBODIMENT

Acid-sensing ion channels (ASICs) are members of the DEG/ENaC superfamily of Na⁺ permeable channels. The ASICs can form homo- and heteromeric channels. They are activated by a drop of pH below 6.8 and desensitize rapidly which has raised the question of their functional role (Akaike et al., 1994). The current invention is based on the finding that ASIC is distributed to regions supporting high levels of synaptic plasticity in the hippocampus and amygdala, thus allowing for novel treatment of anxiety and drug addiction in such a way as to provide useful compositions and pharmaceutical agents which can aid regulation of these physiological responses. The discovery of acid-sensing ion channels (ASICs) provides an opportunity to explore the previously unknown physiological role of neuronal H⁺-evoked currents. The present invention teaches how the acid-sensing ion channel (ASIC) contributes to synaptic plasticity in the hippocampal circuit and areas enriched with strong excitatory synaptic input such as the glomerulus of the olfactory bulb, whisker barrel cortex, cingulated cortex, striatum, nucleus accumbens, amygdala, and cerebellar cortex. The present invention thus teaches the previously unknown effect of ASIC disruption on H⁺-evoked currents in the brain thereby providing methods of treatment for anxiety, anxiety disorders, drug addiction and improved synaptic plasticity for fear conditioning all of which have been attributed to CNS disorders which display neurotransmitter system dysfunction.

Acid-activated cation currents have been detected in central and peripheral neurons for more than 20 years (Gruol et al., 1980; Krishtal and Pidoplichko, 1981). In the central nervous system, they have been observed in the hippocampus (Vyklicky et al., 1990), cerebellum (Escoubas et al., 2000), cortex (Varming, 1999), superior colliculus (Grantyn and Lux, 1988), hypothalamus (Ueno et al., 1992), and spinal cord (Gruol et al., 1980). Currents evoked by a fall in extracellular pH vary in pH sensitivity, with half maximal stimulation ranging from pH 6.8 to 5.6 (Varming, 1999). Despite the wide spread distribution of H⁺-gated currents in the brain, neither their molecular identity nor their physiologic functions are known. In the central nervous system, the function of acid-gated currents in general and H⁺-gated DEG/ENaC channels in particular has remained unknown. The present studies provide insight into the function of these channels in the central nervous system.

Although many central neurons possess large acid-activated currents, their molecular identity and physiologic function have remained unknown. Previous to the discovery of ASIC receptors, the NMDA receptor has been implicated during development in specifying neuronal architecture and synaptic connectivity and may be involved in experience dependent synaptic modifications. NMDA receptors are also thought to be involved in long term potentiation, Central Nervous System (CNS) plasticity, cognitive processes, memory acquisition, retention, and learning. However, activation of the NMDA receptor, which occurs only under conditions of coincident presynaptic activity and postsynaptic depolarization, has displayed significant difficulty. Current medications that are prescribed to either activate or block the NMDA receptor and influence glutamatergic synaptic transmission are poorly tolerated because of severe side effects.

Recently researchers identified a family of cation channels that are gated by reductions in pH. These proteins, called ASICs, are related to amiloride-sensitive epithelial sodium channels (ENaCs) and the degenerin/mec family of ion channels from Caenorhabditis elegans (Waldmann et al., 1997). The acid-sensing DEG/ENaC channels respond to protons and generate a voltage-insensitive cation current when the extracellular solution is acidified.

The current invention relates to the further characterization of the role of ASIC in the brain. According to the invention it was found that although ASIC was present in the hippocampal circuit, it was surprisingly found to be more abundant in several areas outside the hippocampus where ASIC has not been previously definitively identified. The inventors discovered that ASIC was enriched in areas with strong excitatory synaptic input such as the glomerulus of the olfactory bulb, whisker barrel cortex, cingulate cortex, striatum, nucleus accumbens, amygdala, and cerebellar cortex. In addition, it was discovered that the ASIC levels are particularly high in the amygdala. Extracellular acidosis elicited a greater current density in amygdala neurons than hippocampal neurons. Disruption of the ASIC gene eliminated H⁺-evoked currents in the amygdala. In addition, ASIC null mice had impaired hippocampal long term potentiation that was rescued by enhancing NMDA receptor activity with reduced extracellular Mg²⁺ concentration or protein kinase C activation. ASIC1 null mice showed deficits in learning tasks dependent upon brain regions where ASIC1 is normally expressed. The present invention's discovery that ASIC is located in these regions of the cerebral cortex whereby ASIC distribution to these regions of high synaptic plasticity directly implicates the ASIC receptors in the treatment of CNS disorders such as anxiety and addiction.

Thus, the present invention teaches new methods of treatment for anxiety and drug addiction according to the invention's drug screening protocol and pharmacological agents that act as ASIC antagonists and wherein a method of treatment for fear conditioning according to the invention's drug screening protocol and pharmacological agents which act as ASIC agonists will enhance memory, for example, through the neural mechanisms during fear conditioning. Moreover, drugs that effect ASIC can block the damaging affects of extracellular acidosis that induces a desensitization of ASICs and inhibits their physiological function in the brain. The present inventors discovered that extracellular acidosis elicits a greater current density in amygdala neurons than hippocampal neurons therefore disrupting the ASIC 1 gene eliminates H+-evoked currents in the amygdala. Furthermore, the effects of disrupting ASIC are less severe than the effects of disrupting the NMDA receptor, thus medications that affect ASIC activity are better tolerated treatments for anxiety, the neurologic damage that results from drug addiction, and memory loss associated with fear conditioning. These results suggest that acid-activated currents contribute to synaptic plasticity, learning and memory with less severe effects.

The ability of acid to activate three members of the DEG/ENaC channel family, discussed infra, suggest they are responsible for H⁺-gated currents in the central nervous system. Subunits of the DEG/ENaC protein family associate as homomultimers and heteromultimers to form voltage-insensitive channels. Individual subunits share a common structure with two transmembrane domains, intracellular carboxyl- and amino-termini, and a large, cysteine-rich extracellular domain thought to serve as a receptor for extracellular stimuli. Most DEG/ENaC channels are inhibited by the diuretic amiloride. The three mammalian acid-activated DEG/ENaC channels are (1) brain Na⁺ channel 1 (BNC1 (Price et al., 1996), also called MDEG (Waldmann et al., 1996), BNaC1 (García-Añoveros et al., 1997), and ASIC2 (Waldmann and Lazdunski, 1998)), (2) acid sensing ion channel (ASIC (Waldmann et al., 1997b) also called BaNaC2 (García-Añoveros et al., 1997) and ASIC1 (Waldmann and Lazdunski, 1998)), and (3) dorsal root acid sensing ion channel (DRASIC (Waldmann et al., 1997a) also called ASIC3 (Waldmann and Lazdunski, 1998)). BNC1 and ASIC each have alternatively spliced isoforms (BNC1a and 1b, and ASICα and ASICβ)(Chen et al., 1998; Lingueglia et al., 1997; Price et al., 2000). Heterologous expression of most of these subunits generates Na⁺ currents that activate at low extracellular pH and then desensitize in the continued presence of acid (Waldmann and Lazdunski, 1998). Expression of individual subunits and coexpression of more than one subunit generates currents that show distinct kinetics and pH sensitivity.

Based on the transient nature of H⁺-evoked currents in primary cultures of cortical neurons and their inhibition by amiloride, Varming (Varming, 1999) suggested that DEG/ENaC channels and ASIC in particular might be responsible for the endogenous H⁺-gated currents. The pattern of expression was consistent with this idea; ASICα, BNC1a, and BNC1b have transcripts in the central nervous system (García-Añoveros et al., 1997; Waldmann et al., 1997b), whereas DRASIC and ASICβ are expressed primarily in the peripheral nervous system (Chen et al., 1998; Waldmann et al., 1997a). ASIC transcripts were most abundant in the cerebral cortex, hippocampus, cerebellum, and olfactory bulb (García-Añoveros et al., 1997; Waldmann et al., 1997b). A recent study reported that ASIC was inhibited by a peptide toxin from the venom of the South American tarantula Psalmopoeus cambridgei (Escoubas et al., 2000). This peptide also inhibited acid-evoked currents in cultured cerebellar granule cells, further suggesting that ASIC could be a component of these pH-gated currents. Nonetheless, the role of ASIC1 in the amygdala and its contribution to synaptic transmission and physiological significance has previously been unknown.

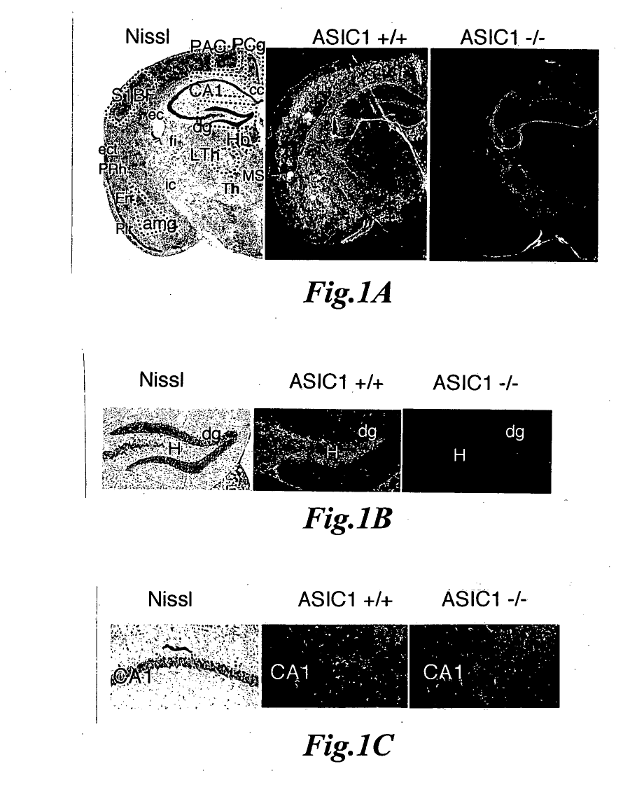

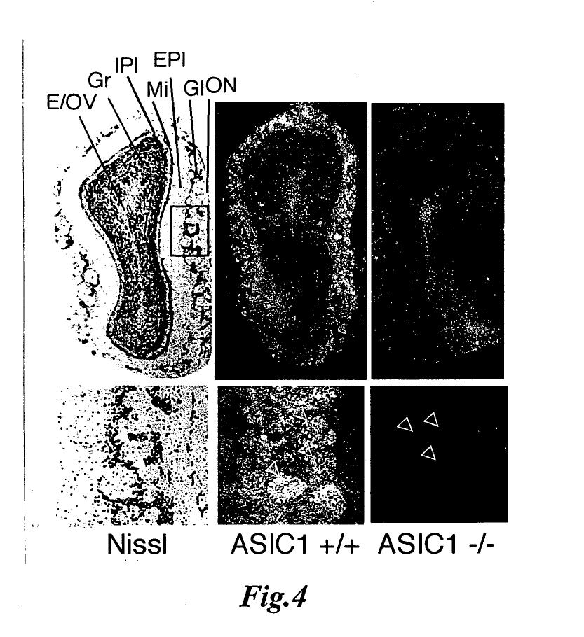

Disrupting ASIC1a in mice eliminated pH 5-evoked current in hippocampal neurons, identifying it as key component of H+-gated currents (Wemmie et al., 2002). The present invention's findings in the hippocampus led inventors to test the hypothesis that H⁺-gated channels influenced other areas of the brain not previously known to be enriched with ASIC. The inventors discovered that ASIC1-null mice were viable, with no obvious anatomic or physiological abnormalities, but they did exhibit deficits in hippocampus-dependent spatial learning and cerebellum-dependent eyeblink conditioning. The degree of 2 0 impairment in cerebellum-dependent eyeblink conditioning was particularly pronounced in ASIC −/− animals and comparable to that observed in Purkinje cell degeneration (pcd) mutant mice (Chen et al., 1996). Those mice exhibit a selective loss of Purkinje cells, the sole output from the cerebellar cortex, and they are functionally equivalent to animals with complete cerebellar cortical lesions. Interestingly, the pcd mice are also ataxic (Chen et al., 1996), as is often the case with impaired cerebellar function (Kim and Thompson, 1997). In contrast, ASIC null mice ambulated normally and demonstrated normal motor learning on the accelerating rotarod. Therefore, the ASIC mutation affects only specific types of learning. These tasks relating to hippocampus-dependent spatial learning and cerebellum-dependent eyeblink conditioning depend on the hippocampus and cerebellum where ASIC is normally expressed ((García-Añoveros et al., 1997; Waldmann et al., 1997b) and FIG. 1) and where H⁺-gated currents have been identified ((Escoubas et al., 2000; Vyklicky et al., 1990) and FIG. 4). Due to the ASIC distribution in the hippocampus being different than previously suggested by others in the art, the inventors searched for distribution elsewhere in the brain.

The inventor's discovery that ASIC contributes to acid activated currents in amygdala neurons led to the claimed invention establishing that ASIC was enriched in these areas with strong excitatory synaptic input. This result greatly expands what has been previously suggested regarding ASICs physiologic contribution to brain function. Moreover, the inventors found that ASIC protein was present in the hippocampus and that acid-activated currents were missing in hippocampal neurons of ASIC −/− mice; these results indicated that ASIC is a key component of the channels that produce H⁺-gated currents. These data provide, at least in part, a molecular identity to the H⁺-gated currents that for many years have only been observed only in central neurons (Escoubas et al., 2000; Grantyn and Lux, 1988; Ueno et al., 1992; Varming, 1999; Vyklicky et al., 1990). Prior to the inventor's discovery it was postulated that these currents were only present in the hippocampal circuit. Therefore, it was a surprise that hippocampal neurons from ASIC null animals had no detectable transient acid-evoked current as it was believed this was the only area they were present in. There are at least two potential explanations. First, ASIC is the only DEG/ENaC subunit responsible for the H⁺-gated currents. Second, ASIC combines with BNC1a or other DEG/ENaC subunits to generate current, but their function depends on the presence of ASIC for some step in biosynthesis or function. Nevertheless, the present invention teaches that ASIC is required for normal synaptic plasticity which provides new methods of treatment for anxiety, drug addiction and fear conditioning which were not previously known.

The most plausible mechanism of learning-related plasticity in the cerebellar cortex is long-term depression (LTD) between granule and Purkinje cells (Hansel et al., 2001; Maren and Baudry, 1995; Mauk et al., 1998). These cells represent a key point of convergence between the neural pathways that carry the conditioned and unconditioned stimuli. Interestingly, mature Purkinje cells do not express functional NMDA receptors (Farrant and Cull-Candy, 1991) (Llano et al., 1991). However, LTD does require post-synaptic membrane depolarization and increased post-synaptic Ca²⁺ concentrations (Daniel et al., 1998; Linden, 1994), features shared between cerebellar LTD and hippocampal LTP. As the inventors hypothesized for the hippocampus, ASIC contributes to elevations in post-synaptic Ca²⁺ concentration directly, or indirectly through membrane depolarization. A reduction in either of these processes will impair synaptic plasticity and memory formation in the cerebellum. In addition, ASIC −/− animals prove to be a useful model to further ascertain cerebellar function.

Thus according to the invention, ASIC offers a novel pharmacological target for modulating excitatory neurotransmission. Involvement of ASIC in synaptic plasticity suggests that its activity can be manipulated for methods of treatment of anxiety, drug addiction, and fear conditioning and for pharmacological purposes because ASIC enables H⁺-gated currents which enable synaptic transmission that contributes to such neural behaviors as anxiety and addiction. In addition, ASIC can be inhibited to minimize the adverse consequences of acidosis. ASIC antagonists provide a way to dampen excitatory transmission without inhibiting other key components of the system. ASIC disruption has no drastic consequences on animal development, viability, or baseline synaptic transmission. In contrast, currently used treatments, such as benzodiazepines, have highly addictive qualities and can lead to an undesirable increase in the targeted behavior. Protocols for identifying potential therapeutic agents for the treatment of anxiety, drug addiction, and fear conditioning will offer rich opportunities for improved treatments and new targets for pharmacotherapy.

The treatment of anxiety is just one of the conditions of the CNS that ASIC antagonists can assist with through modulation of the acid-sensing ion channel. Anxiety is a normal emotional feeling, in appropriate situations, related to different situations of threat or fear, but excessive anxiety and anxiety in inappropriate situations can be disabling. External threat is experienced as a fear whereas obscure and unidentified feeling of threat may be experienced as anxiety. When anxiety persists it can develop into a pathological disorder. Anxiety disorders are divided more specifically in diagnostic disorders e.g., panic disorder, phobias, and GAD. GAD is a chronic illness associated with excessive anxiety and worry lasting for at least six months. In addition, the anxiety and worry are associated with restlessness, fatigue, difficulties in concentrating or mind going blank, irritability, muscle tension, and sleeping disturbances. The symptoms may be triggered by different events of life, and the control of anxiety is very difficult for the patient.

Anxiety is associated with a bilateral increase in blood flow in a discrete portion of the anterior end of each temporal lobe. The present invention has discovered that ASIC is abundant in the lateral, basolateral and central nuclei of the amygdala thereby having a direct effect on amygdala-dependent behavior such as anxiety. Utilization of the channels induces transient currents that participate in synaptic function thus proton-activated currents in the neurons studied are mediated by the ASIC.

Anxiety is currently treated with benzodiazepines, SSRI's and buspirone, which are not optimal treatments due to adverse drug reactions and their efficacy profiles. Often it is observed that patients with anxiety disorders have decreased sensitivity to benzodiazepines. Moreover, relapse of the disease, different kinds of withdrawal effects, development of tolerance, as well as relapse and recurrence, often happen when traditional anxiolytics are used. For example, to avoid withdrawal effects, doctors usually gradually taper the dosage of the medicine (i.e. gradually diminish its daily dosage) before the treatment may be stopped. Patients tend to develop tolerance to those traditional compounds as well. Development of tolerance occurs when, for example, a patient requires greater quantities of a compound over time to achieve the same therapeutic effect. Therefore, there is a need in the art to better understand and treat anxiety disorders as taught in the present invention.

The invention relates to novel methods for the treatment, prevention, inhibition and amelioration of conditions in patients in need thereof including anxiety, generalized anxiety disorder, panic anxiety, obsessive compulsive disorder, social phobia, performance anxiety, post-traumatic stress disorder, acute stress reaction, adjustment disorders, hypochondriacal disorders, separation anxiety disorder, agoraphobia and specific phobias. Such patients may be those presently experiencing the anxiety-related symptoms or conditions of these disorders or those subject to such occurrences. Specific anxiety related phobias which may be treated with these methods are those commonly experienced in clinical practice including, but not limited to, fear of animals, insects, storms, driving, flying, heights or crossing bridges, closed or narrow spaces, water; blood or injury, as well as extreme fear of inoculations or other invasive medical or dental procedures. In order to elicit their behavioral effects, the compounds of the invention will ideally be brain-penetrant; in other words, these compounds will be capable of crossing the so-called “blood-brain barrier”. Preferably, the compounds of the invention will be capable of exerting their beneficial therapeutic action following administration by the oral route.

In the treatment of psychiatric disorders with a chronic course, such as anxiety, it is important to prevent the relapse and recurrence of the disease. After the acute treatment phase, the improved condition can be maintained, and relapses can thus be prevented by continuing the treatment in those who have responded to the treatment or who have reached remission during it. After the continuation treatment phase, when recovery has been reached, the disease can be prevented by continuing the treatment further by the so-called maintenance treatment, during which the daily dosage may be decreased, for example, to a half from the original. Furthermore, sufficient efficacy in relapse and recurrence prevention are important qualities of the present invention's compositions and treatment.

The treatment of addiction is also improved with ASIC antagonists. Drugs that modify human behavior act by modifying transmission at synaptic junctions in the brain. The present invention describes how ASICs function as a key component of acid-activated currents implicating these currents in processes underlying synaptic plasticity which plays a role in the development of addiction. Addiction, defined as the repeated compulsive use of a substance despite negative consequences, can be produced by a variety of drugs. Not surprisingly, addiction is associated with the reward system, and particularly with the nucleus accumbens, located at the base of the striatum and the mesocortical dopaminergic neurons that project from the midbrain to this nucleus and the frontal cortex. Animals will press bars and perform other tasks to receive injections of addicting drugs though chronically implanted catheters. The best-studied addictive drugs are opiates such as morphine and heroin, cocaine, amphetamine, ethyl alcohol, and nicotine. All of these affect the brain in different ways, but all have in common the fact that they increase the amount of dopamine available to act on D₃ receptors in the nucleus accumbens. Thus, acutely they stimulate the reward system of the brain. Long term, however, addiction involves the development of tolerance, i.e. the need for increasing amounts of a drug to produce a “high”. Withdrawal produces psychologic and physical symptoms. The causes of tolerance and withdrawal symptoms are as yet not fully understood. One of the characteristics of addiction is the tendency of addicts to relapse after treatment. For opiate addicts, for example, the relapse rate in the first year is about 80%. Relapse often occurs upon exposure to sights, sounds, and situations that were previously associated with drug use. The medial-prefrontal cortex and hippocampus, and the amygdala send excitatory glutaminergic fibers to the nucleus accumbens, and it is expected that activity in these inputs contributes to the relapses produced by environmental cues and memories. In addition, some cases of epilepsy can be a hybrid of subtypes, while others defy precise categorization. Nonetheless, elimination of ASIC activity has been found to block the damaging effects that occur during addiction.

The present invention also describes that the acid-sensing ion channel 1 (ASIC1) protein is preferentially distributed to brain regions with strong excitatory synaptic input. Because ASIC1 is abundant in the lateral and basolateral nuclei of the amygdala, the inventors' found that the freezing deficit presented when tested for cued fear conditioning in the ASIC1-null mice is due to the impaired learning, especially because baseline fear on the elevated plus maze is intact. Nonetheless, ASIC1 is also expressed in other regions of the fear circuit, for example, the cingulate cortex, nucleus accumbens, and central nucleus of the amygdala, structures through to contribute to the emotional importance of external stimuli and/or expression of fear (Cardinal et al., 2002). Thus, ASIC1 also affects multiple brain regions underlying the acquisition and expression of the associative learning during fear conditioning.

Further, there are many memory-related conditions for which therapeutic treatments are under investigation, such as methods to enhance memory or to treat memory dysfunction. For example, memory dysfunction is linked to the aging process, as well as to neurodegenerative diseases such as Alzheimer's disease. In addition, memory impairment can follow head trauma or multi-infarct dementia. In the present invention, the ASIC receptor enhances learning, memory, and the neural mechanisms of fear conditioning.

The present invention thus seeks to provide a safer and improved ASIC receptor antagonist for general pharmaceutical use to treat anxiety, drug addiction, and other conditions associated with acidosis. In addition, ASIC receptor agonists will allow treatment and preventative uses for conditions associated with fear conditioning that is linked to synaptic plasticity in the amygdala.

Accordingly, the present invention provides a method for screening new therapeutic agents for the treatment of anxiety or drug addiction by assaying for the agents ability to act as an antagonist or in the case of treatment for fear conditioning to act as an agonist of the acid-sensing ion channel family. The assay comprises administering the composition to be screened to cells expressing acid-gated channels and then determining whether the composition has modulates the acid-sensing channels of the DEG/ENaC family. The determination can be performed by analyzing whether a current is generated in cells containing these channels in the presence of the composition and the acid. This current can be compared to that sustained by the FMRFamide and FMRFamide-related peptides.

In addition to the ASIC channels, it is expected that FMRFamide or FMRFamide related peptides will potentiate acid-evoked activity of other members of the DEG/ENaC cation channel family. The determination of enhancement or inhibition can be done via electrophysical analysis. Cell current can be measured. Alternatively, any indicator assay which detects opening and/or closing of the acid-sensing ion channels can be used such as voltage-sensitive dyes or ion-sensitive dyes. An assay which caused cell death in the presence of the peptide, or agonist, would be the most definitive assay for indicating potentiation of the channels. Assays which could measure binding of FMRFamide and related peptides to the channels could identify binding of agonists, antagonists, and modulators of binding. One of ordinary skill in the art would be able to determine or develop assays which would be effective in finding compositions which effect the acid-sensory ion channels. A composition which activates or inactivates the transient or sustained current present when acid or a related peptide activate the acid-sensing ion channels should be useful as a pharmacological agent. The screening can be used to determine the level of composition necessary by varying the level of composition administered. The composition can be administered before or after addition of the acid or a related peptide to determine whether the composition can be used prophylactically or as a treatment for enhanced synaptic plasticity, learning or memory. One of ordinary skill in the art would be able to determine other variations on the assay(s).

Suitable formulations for parenteral administration of a therapeutically effective amount of a pharmaceutical composition incorporating an acid sensing ion channel (ASIC) include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example, ethyl oleate or triglycerides. Aqueous injection suspensions may contain substances which increase the viscosity of the suspension, include for example, sodium carboxymethyl cellulose, sorbitol and/or dextran, optionally the suspension may also contain stabilizers. In addition to administration with conventional carriers, active ingredients may be administered by a variety of specialized delivery drug techniques which are known to those of skill in the art. The following examples are given for illustrative purposes only and are in no way intended to limit the invention.

Compositions which bind to the channels can be identified or designed (synthesized) based on the disclosed knowledge of potentiation of the channels and determination of the three-dimensional structure of the channels. These compositions could act as agonists, antagonists, or modulators effecting synaptic plasticity, learning, memory or other physiological responses.

In general, in addition to the active compounds, i.e. the ASIC agonists and antagonists, the pharmaceutical compositions of this invention may contain suitable excipients and auxiliaries which facilitate processing of the active compounds into preparations which can be used pharmaceutically. Oral dosage forms encompass tablets, dragees, and capsules. Preparations which can be administered rectally include suppositories. Other dosage forms include suitable solutions for administration parenterally or orally, and compositions which can be administered buccally or sublingually.

The pharmaceutical preparations of the present invention are manufactured in a manner which is itself well known in the art. For example the pharmaceutical preparations may be made by means of conventional mixing, granulating, dragee-making, dissolving, lyophilizing processes. The processes to be used will depend ultimately on the physical properties of the active ingredient used.

Suitable excipients are, in particular, fillers such as sugars for example, lactose or sucrose mannitol or sorbitol, cellulose preparations and/or calcium phosphates, for example, tricalcium phosphate or calcium hydrogen phosphate, as well as binders such as starch, paste, using, for example, maize starch, wheat starch, rice starch, potato starch, gelatin, gum tragacanth, methyl cellulose, hydroxypropylmethylcellulose, sodium carboxymethylcellulose, and/or polyvinyl pyrrolidone. If desired, disintegrating agents may be added, such as the above-mentioned starches as well as carboxymethyl starch, cross-linked polyvinyl pyrrolidone, agar, or alginic acid or a salt thereof, such as sodium alginate. Auxiliaries are flow-regulating agents and lubricants, for example, such as silica, talc, stearic acid or salts thereof, such as magnesium stearate or calcium stearate and/or polyethylene glycol. Dragee cores may be provided with suitable coatings which, if desired, may be resistant to gastric juices.

For this purpose concentrated sugar solutions may be used, which may optionally contain gum arabic, talc, polyvinylpyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, dyestuffs and pigments may be added to the tablet of dragee coatings, for example, for identification or in order to characterize different combination of compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, liquid paraffin, or liquid polyethylene glycols. In addition stabilizers may be added. Possible pharmaceutical preparations which can be used rectally include, for example, suppositories, which consist of a combination of the active compounds with the suppository base. Suitable suppository bases are, for example, natural or synthetic triglycerides, paraffinhydrocarbons, polyethylene glycols, or higher alkanols. In addition, it is also possible to use gelatin rectal capsules which consist of a combination of the active compounds with a base. Possible base material includes for example liquid triglycerides, polyethylene glycols, or paraffin hydrocarbons.

Suitable formulations for parenteral administration include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example, ethyl oleate or triglycerides. Aqueous injection suspensions may contain substances which increase the viscosity of the suspension, include for example, sodium carboxymethyl cellulose, sorbitol and/or dextran, optionally the suspension may also contain stabilizers.

In addition to administration with conventional carriers, active ingredients may be administered by a variety of specialized delivery drug techniques which are known to those of skill in the art. The following examples are given for illustrative purposes only and are in no way intended to limit the invention.

In conclusion, these results indicate that acid-activated channels influence synaptic plasticity, learning and memory. Further, elucidation of the mechanisms that control ASIC activity and the connection between H⁺-gated channels and behavior should provide new insight and treatments for synaptic function and the processes that underlie synaptic plasticity, learning and memory.

The method and means of accomplishing each of the above objectives will become apparent from the detailed description of the invention which follows. Additional objectives and advantages of the invention will be set forth in part in the description that follows, and in part will be obvious from the examples, or may be learned by the practice of the invention. The objectives and advantages of the invention will be obtained by means of the instrumentalities and combinations, particularly pointed out in the claims of the invention.

It is to be understood that both the foregoing general description and the following detailed description are exemplary and explanatory only and are not restrictive of the invention, as claimed.

### REFERENCES

Allen J J, et al. (2002) Modulation of ASIC channels in rat cerebellar Purkinje neurons by ischaemia-related signals. J Physiol (Lond) 543:521-529.
Alvarez de la Rosa D, et al. (2003) Distribution, subcellular localization and ontogeny of ASIC1 in the mammalian central nervous system. J Physiol (Lond) 546:77-87.
Benson C J, et al. (2002) Heteromultimerics of DEG/ENaC subunits form H⁺-gated channels in mouse sensory neurons. Proc Natl Acad Sci USA 99:2338-2343.
Bianchi L, et al. (2002) Protons at the gate: DEG/ENaC ion channels help us feel and remember. Neuron 34:337-340.
Bolshakov K V, et al. (2002) Characterization of acid-sensitive ion channels in freshly isolated rat brain neurons. Neuroscience 110:723-730.
Cardinal R N, et al. (2002) Emotion and motivation: the role of the amygdala, ventral striatum, and prefrontal cortex. Neurosci Biobehav Rev 26:321-352.
Escoubas P, et al. (2000) Isolation of a tarantula toxin specific for a class of proton-gated Na⁺ channels. J Biol Chem 275:25116-25121.
Faneslow M S, et al. (1999) Why we think plasticity underlying pavlovian fear conditioning occurs in the basolateral amygdala. Neuron 23:229-232.
Fox K (2002) Anatomical pathways and molecular mechanisms for plasticity in the barrel cortex. Neuroscience 111:799-814.
Garcia-Añoveros J, et al. (1997) BNaC1 and BNaC2 constitute a new family of human neuronal sodium channels related to degenerins and epithelial sodium channels. Proc Natl Acad Sci USA 94:1459-1464.
Gruol D L et al. (1980) Hydrogen ions have multiple effects on the excitability of cultured mammalian neurons. Brain Res 183:247-252.
Hyman S E et al. (2001) Addiction and the brain: the neurobiology of compulsion and its persistence. Nat Rev Neurosci 2:695-703.
Immke D C et al. (2001) Lactate enhances the acid-sensing Na⁺ channel on ischemia-sensing neurons. Nat Neurosci 4:869-870.
Krishtal O A et al. (1981) A “receptor” for protons in small neurons of trigeminal ganglia: possible role in nociception. Neurosci Lett 24:243-246.
Krishtal O A et al. (1987) Rapid extracellular pH transients related to synaptic transmission in rat hippocampal slices. Brain Res 436:352-356.
Lingueglia E et al. (1997) A modulatory subunit of acid sensing ion channels in brain and dorsal root ganglion cells. J Biol Chem 272-29778-29783.
Lowry O H et al. (1972) A multistep metabolite assay (for inorganic phosphate). In: A flexible system of enzymatic analysis, Ed 3 (_____, _____, eds), pp 86-92. New York: Academic.
McKernan M G et al. (1997) Fear conditioning induces a lasting potentiation of synaptic current in vitro. Nature 390:607-611.
Olson E H (1998) An acid sensing ion channel (ASIC) localizes to small primary afferent neurons in rats. Neuron 9:1109-1113.
Price M P et al. (2000) The mammalian sodium channel BNC1 is required for normal touch sensation. Nature 407:1007-1011.
Price M P et al. (2001) The DRASIC cation channel contributes to the detection of cutaneous touch and acid stimuli in mice. Neuron 32:1071-1083.
Rogan et al. (1997) Fear conditioning induces associative long-term potentiation in the amygdala. Nature 390:604-607.
Shepherd G M et al. (1998) Olfactory bulb. In: The synaptic organization of the brain, Ed 4 (Shepherd G M, ed), pp 159-204. New York: Oxford UP.
Waldmann R et al. (1997) A proton-gated cation channel involved in acid-sensing. Nature 386:173-177.
Welsh et al. (2002) Biochemical basis of touch perception: mechanosensory function of degenerin/epithelial Na⁺ channels. J Biol Chem 27:2369-2372.
Wemmie, J A, et al. (2002) The acid-activated ion channel ASIC contributes to synaptic plasticity, learning, and memory. Neuron 34:463-477.

All publications, patents and patent applications identified above are herein incorporated by reference, as though set forth herein in full. The invention being thus described, it will be apparent to those skilled in the art that the same may be varied in many ways without departing from the spirit and scope of the invention. Such variations are included within the scope of the following claims.

### EXAMPLES

To understand the role of acid-gated currents in central neurons in general, and the role of ASIC in particular, the inventors generated mice with a targeted disruption of the ASIC gene. The inventors then examined how ASIC contributes to neuronal acid-gated currents and to synaptic function and behavior.

Materials and Methods

Generation of ASIC Knockout Mice

The results were determined by the generation of ASIC knockout mice as described in the following model. This animal model can be used for predicting success in humans. ASIC knockout mice were generated by homologous recombination in embryonic stem cells using an approach similar to that previously reported (Price et al., 2000). A 17 kb genomic clone containing a portion of the ASIC gene was obtained by screening a lambda bacteriophage library of mouse strain SV129 genomic DNA. The wild-type locus, targeting vector and targeted locus are shown schematically in FIG. 1A. In the knockout allele, a PGK-neo cassette replaces the first exon of the ASIC gene and approximately 400 bp of upstream sequence. The deleted exon encodes amino acids 1-121 of mASICα. The neo cassette introduced a new Sac I restriction enzyme site, which was used to screen for targeted integration of the vector. The wild-type and knockout alleles were identified in stem cell clones and in mice by Southern blotting Sac I digested genomic DNA with oligo-labeled cDNA probes corresponding to a 1 kb region that flanks the sequence contained in the targeting vector or with a cDNA probe corresponding to the disrupted sequence. Genotyping was performed by isolating genomic DNA from tail snippets by PCR using the following primers: wild type allele (5′-CCGCCTTGAGCGGCAGGTTTAAAGG-3′, SEQ ID NO:1; 5′-CATGTCACCAAGCTCGACGAGGTG-3′, SEQ ID NO:2), knockout allele (5′-CCGCCTTGAGCGGCAGGTTTAAAGG-3′, SEQ ID NO:3; 5′TGGATGTGGAATGTGTGCGA-3′, SEQ ID NO:4). Northern blotting was performed using the disrupted exon of ASIC as a cDNA oligo-labeled probe against equivalent amounts of total brain RNA. BNC1 RNA expression levels were determined using a probe described previously (Price et al., 2000). Brain histology was performed on mouse brains removed following halothane anesthesia and whole body perfusion with 4% formaldehyde. Brains were fixed overnight, embedded in paraffin, cut into 6 μm sections and stained for Niss1 substance with crystal violet acetate.

Antibody

Polyclonal antiserum (MTY19) was raised in rabbits against the 22 amino acid peptide from the C terminus of ASIC1, MTYAANILPHEPARGTFEDFTC (SEQ ID NO:5), coupled to keyhole limpet hemocyanin (Pocono, Canadensis, Pa.). The IgG fraction was purified using the Econo-Pac serum IgG purification kit (Bio-Rad, Richmond, Calif.). Next, an Affi-gel 15 Gel (Bio-Rad) coupled to the nonspecific peptide GTCNAVTMDSDF (SEQ ID NO:6) was used to adsorb additional nonspecificity for 1 hour at 4° C. (Labquake shaker; Labindustries, Berkeley, Calif.). To adsorb additional nonspecific components of the sera, the inventors used protein extract obtained from ASIC1 knock-out brains coupled to Affi-gel 15 (Bio-Rad), although this step later proved unnecessary. The eluate from these columns was then bound for 4 hours at 4° C. to the immunogenic peptide crosslinked to Affi-gel 15. The specific antibody was eluted with 50 mM glycine/HCl at a pH of 2.5, 150 mM NaCl, neutralized with 1 M Tris at a pH of 10.4, and adjusted to 1% BSA, 0.2% NaN³ for storage at 4° C.

Immunohistochemistry

Coronal brain slices (7.5 μm) were cut on a cryostat (CM 1990; Leica Bannockburn, Ill.) from tissue that was fresh-frozen on dry ice and embedded in Tissue Freezing Medium (Electron Microscopy Sciences, Fort Washington, Pa.). The slices were dried overnight and hydrated with PBS. They were then fixed in PBS with 5% formaldehyde, 4% sucrose for 15 minutes, followed by 0.25% Triton X-100 in PBS for 5 minutes at room temperature. After two rinses with PBS, endogenous peroxidase activity was quenched with 3% H₂O₂ for 30 minutes. This was followed with three 5 minute washes with PBS and blocking with Tris/NaCl/blocking reagent buffer (TNB) (TSA Fluorescence Systems, PerkinElmer Life Sciences, Boston, Mass.) for 30 minutes. Purified MTY19 (1:50 in TNB) or anti-calbindin D-28K (Chemicon International, Temecula, Calif.) was added and allowed to incubate for 2 hours. After three 5 minute washes with PBS, α-rabbit IgG-horse radish peroxidase (HRP) (Amersham Biosciences, Piscataway, N.J.) was used as a secondary antibody at 1:200 for 1 hour at 37° C. After another three 5 minute washes with PBS, the signal was amplified by incubating in tyramide solution (TSA Fluorescence Systems, Perkin-Elmer Life Sciences, Boston, Mass.) for 10 minutes at room temperature. Finally, the slices were washed three more times for 5 minutes with PBS, mounted with Vectashield (Vector Laboratories, Burlingame, Calif.), and visualized by Bio-Rad MRC 1024 confocal microscope, or Olympus BX-51 epifluorescence microscope (Melville, N.Y.) equipped with Spot RT Slater (Diagnostic Instruments, Sterling Heights, Mich.). The specific ASIC1 immunostaining was lost in paraffin-embedded tissue and when sections were prepared from brain perfused with formalin in vivo before sectioning.

Immunoblotting

From 500 μm Vibratome cut slices (Pelco, Redding, Calif.), the amygdala, CA1, CA3, posterior cingulated, posterior association cortex, habenula, and thalamus were dissected according to regions surrounded by a dashed line in FIG. 1. Tissue homogenate was also obtained from the whole brain and cerebellum. The tissue was homogenized in PBS with protease inhibitors (aprotinin 40 μg/ml, leupeptin 40 μg/ml, pepstatin A 20 μf/ml, PMSF 40 μg/ml, and EDTA 2 mM) using a 1 ml Dounce homogenizer (Wheaton, Millville, N.J.). The homogenate was cleared of large unground particles with a 10 minute centrifugation at 3500 rpm (5415C; Eppendorf Hamburg, Germany). Membrane proteins were precipitated at 70,000 rpm for 30 minutes (TL-100; Beckman, Fullerton, Calif.). The pellet was resuspended in PBS with protease inhibitors. All steps in sample preparation were performed on ice or at 4° C. Protein concentration was determined (Lowry and Passanneau, 1972), and 100 μg was run on 8% acrylamide gel and Western blotted. The blot was first probed with MTY19 serum at 1:15,000, followed by α-rabbit IgG-HRP (Amersham Biosciences) at 1:10,000. The signal was detected by enhanced chemiluminescence (Pierce, Rockford, Ill.).

Whole-Cell Voltage-Clamp Experiments

Mouse hippocampal cultures were generated from postnatal day 1-2 pups as described previously (Wemmie et al., 2002). Amygdala cultures were generated by the same method except that the amygdala was dissected from 1 mm coronal sections using the external capsule as a landmark to define the borders of the lateral and basolateral amygdala. Culture medium contained insulin, transferring, and sodium selenite (I-1884, Sigma, St. Louis, Mo.), resuspended in 50 ml H₂O, 2.5 μl/ml of medium. Whole-cell patch-clamp recordings were performed on neurons from at least two different preparations that were cultured for 1-2 weeks. Electrodes (3-5 mΩ) were filled with intracellular solutions containing (in mM): 120 KC1, 10 NaCl, 2 MgCl₂, 5 EGTA, 10 HEPES, and 2 ATP. The pH was adjusted to 7.2 with KOH. Extracellular solutions contained (in mM): 128 NaCl, 2 CaCl₂, 1 MgCl₂, % 0.4 KCl, 5.55 glucose, 10 HEPES, and 10 MES. To inhibit spontaneous activity, 0.5 μM tetrodotoxin, 5 μM CNQX, 15 μM bicuculline methiodide, and 25 μM DL-2-amino-5-phosphonovaleric acid were added to the extracellular solutions. The pH was adjusted with tetramethylammonium hydroxide (TMA-OH) and the osmolarity adjusted with TMA-C1. Neurons were held at −80 mV for recording, and extra-cellular pH was 7.4 unless otherwise indicated. All chemicals were obtained from Sigma.

Elevated-Plus Maze

A maze was constructed from stainless steel with a Plexiglas base (36 inches tall) and two pair of arms (2×11⅝ inches) intersecting at right angles. One pair of arms was closed and had six inner walls on three sides. The two open arms lacked walls. A 2×2 inch intersection connected the four arms. Naïve mice (+/+, n=11; −/−, n=11) were placed onto the center of the maze and allowed 5 minutes to roam freely. Activity was recorded by a video camera suspended above the maze. A trained technician blinded to genotype recorded the time each animal spent in the closed arms, open arms, and stationary in the corner of the closed arms. The number of entries into the open central intersection was also determined. Statistical significance was tested with a two-sample t test.

Auditory Fear Conditioning

On day 1, naïve mice (+/+, n=7; −/−, n=9) were placed in a conditioning chamber (Lafayette Instrument, Lafayette, Ind.). After 3 minutes, they were presented with a tone (80 dB, 20 sec) that co-terminated with an electric foot-shock (1 mA, 1 sec). A total of seven pairings of the tone and shock were delivered, separated by 1 minute intervals. Mice were then returned to their home cage. On day 2, to minimize freezing to context, the lights were dimmed, burgundy poster board was used to change the color of the back wall and ceiling, a wire mesh floor grate was inserted, white bench paper was placed under the floor grid, and the paper was dabbed with 1 drop of peppermint extract. The animals were placed in the conditioning chamber, observed for 3 minutes, and then presented with the same tone continuously for 6 minutes, minus the foot-shock. Freezing (defined as a crouched posture and an absence of movement) during 1 minute intervals was quantified from videotapes by a trained observer blinded to genotype. Three −/− mice and one +/+ mouse were excluded from the training data because they climbed onto the wall of the chamber during at least one interval. Although this did not interfere with the conditioning protocol, it did interfere with scoring and disqualified them from the ANOVA with repeated measures. One +/+ mouse was excluded from the study because its tail was inadvertently pinched as it was being placed into the chamber. Another +/+ mouse was excluded because its freezing response was >3 SD from the mean. The context fear conditioning protocol was similar, except on day 1, the mice received three shocks and no tone was presented (+/+, n=8; −/−, n=7). On day 2, the same chamber was used without changing the context.

Results and Discussion

ASIC1 Immunolocalization in the Brain

Previous in situ hybridization studies suggested that ASIC1 transcripts were abundant in layers CA1 through CA4 of the hippocampus (García-Añoveros et al., 1997; Waldmann et al., 1997). In addition, the inventors have shown previously that disrupting ASIC1 impairs Schaffer collateral-CA1 LTP and adversely affects spatial learning (Wemmie et al., 2002). Therefore, the inventors determined where in the hippocampal circuit ASIC1 protein was located. An affinity-purified rabbit polyclonal antibody against the C-terminal 22 amino acids of mouse ASIC1 was used to immunolabel coronal sections of mouse brain (FIG. 1A). As a control, the inventors used ASIC1 −/− brains. In the hippocampus, the hilus (polymorphic layer) of the dentate gyrus showed the most prominent ASIC1 staining. This region is occupied by inhibitory and excitatory interneurons as well as mossy fibers and CA3 dendrites (FIG. 1B).

In contrast, ASIC1 immunostaining in CA1 and CA2 was relatively weak (FIG. 1A, C). Others have suggested that epitope masking may obscure ASIC1 detection in the brain (Olson et al., 1998). To address this possibility, the inventors also immunoblotted protein obtained from the dentate gyrus and CA1. Although ASIC1 was detected, it was dramatically reduced in CA1 compared with the dentate gyrus (FIG. 1D). Thus, although ASIC1 may have important effects on CA1 function (Wemmie et al., 2002), the amount of protein in this region may be sparse relative to other areas.

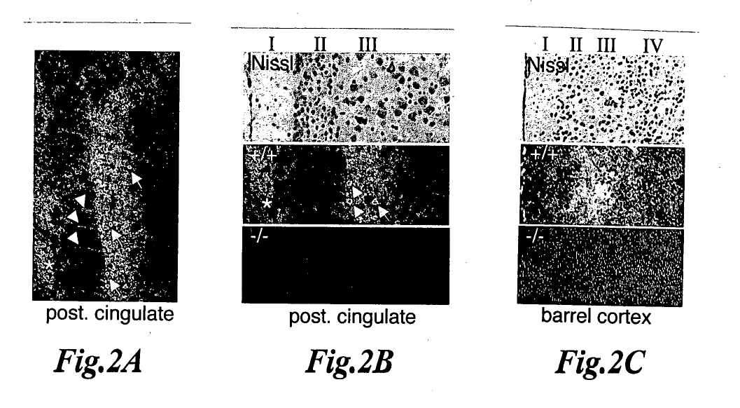

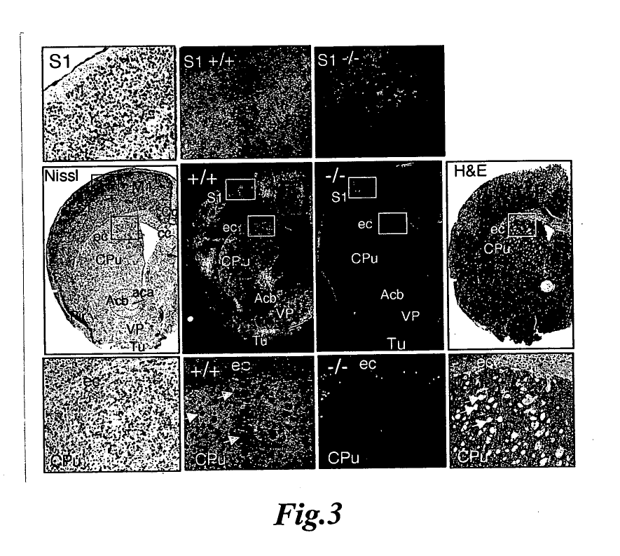

Because ASIC1 distribution in the hippocampus was different than anticipated, the inventors proceeded to determine its distribution elsewhere in the brain. Previous studies reported that ASIC1 mRNA was elevated in the cerebral cortex (García-A\ddot{n}overos et al., 1997; Waldmann et al., 1997; Waldmann et al., 1997). Consistent with those reports, the present invention found abundant ASIC1 protein in a number of specific cortical regions (FIGS. 1A, 2, 3). ASIC1 staining was evident in the anterior and posterior cingulated cortex (FIGS. 1A, 2A, B). The sensory and motor cortices were also immunopositive (FIGS. 1A, 3). A subdomain of the sensory cortex in which ASIC1 staining was prominent was the whisker barrel field (FIGS. 1A, 2C), an area that has served as a valuable model system for analyzing cortical plasticity (for review, see Fox, 2002). In contrast, ASIC1 immunostaining was low in the ectorhinal, perirhinal, and piriform cortex (FIGS. 1A, 3).

ASIC1 immunostaining in sensorimotor and cingulated cortex tended to be elevated in layer III. For example, in the posterior cingulated, immunolabeling could be seen on pyramidal cell bodies in layer III (FIG. 2A, B, arrows) and also in layer I near the brain surface FIG. 2A, asterisk). In the present invention it was consistently observed stripes of staining perpendicular to the cortical layers and extending between layers I and III, possibly caused by apical dendrites extending from pyramidal neurons in the deeper layers (FIG. 2A, arrowhead). ASIC1 staining in barrel and motor cortex was also preferentially distributed to layer III (FIGS. 1A, 2C). The significance of layer III specificity is not clear, although it is interesting to note that an NMDA receptor-dependent form of LTP in this layer has been implicated in barrel cortex function (Fox, 2002).

In addition to the cortex, the inventors observed strong ASIC1 staining in certain subcortical structures, including the basal ganglia (FIG. 3). ASIC1 labeling was readily apparent in the striatum, in which it was distributed in gray matter, and was slightly more abundant dorsally and laterally (FIG. 3), regions that preferentially receive sensorimotor cortical input. The strong signal in gray matter of the striatum contrasted sharply with weak white matter staining, giving the ASIC1 distribution a dappled appearance (FIG. 3). The present invention further observed strong ASIC1 in the ventral pallidum, olfactory tubercle, and nucleus accumbens (FIG. 3). The basal ganglia serve an important role in voluntary movement. The striatum and nucleus accumbens may also contribute to motivation and appetitive behavior and have been linked to addiction in humans (for review, see Cardinal et al. 2002; Hyman and Malenka, 2001). Yet, ASIC1 knock-out mice performed normally on the accelerating Rotarod (Wemmie et al., 2002) and displayed normal activity on the elevated plus maze (see below). Nevertheless, the high level of ASIC1 in the striatum suggests that given the appropriate challenge, ASIC1-null mice exhibit abnormal striatum-dependent behavior.

In contrast to the basal ganglia, ASIC1 immunostaining in the thalamus was rather weak, with the exception of the habenula and the medial septal nuclei (FIG. 1A). The significance of the selective distribution between subcortical structures is not yet clear.

The present invention also tested for ASIC1 protein in the olfactory bulb, because ASIC1 mRNA was reported to be elevated there (Waldmann et al., 1997). The inventors discovered ASIC1 protein localized preferentially to the glomerular layer and most evident within glomeruli (FIG. 4, arrows). Immunolabeling of periglomerular cells was less intense, causing the striking glomerular pattern to stand out (FIG. 4). Glomeruli provide a site for synaptic contact between olfactory sensory neurons are continuously replaced throughout life, synapses in the glomerulus undergo constant remodeling (for review, see Shepherd and Greer, 1998). This high degree of plasticity is unique in the mammalian brain. The strong ASIC1 signal in the glomeruli is consistent with the ability of ASIC1 to affect synaptic function (Wemmie et al., 2002).

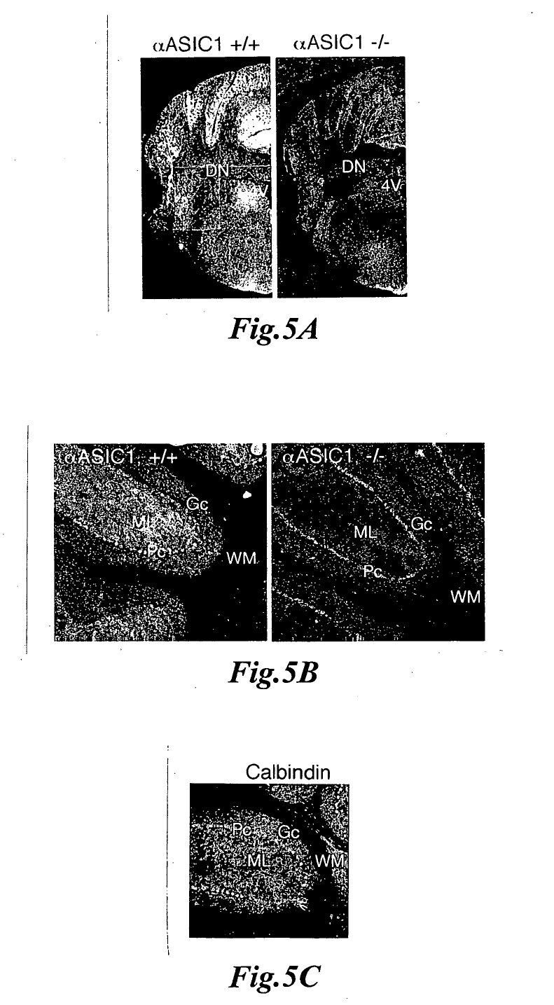

The cerebellum contains abundant ASIC1 mRNA (García-Añoveros et al., 1997; Waldmann et al., 1997), and eyeblink conditioning studies suggested that ASIC1 may have important effects on cerebellum-dependent learning (Wemmie et al., 2002). In the cerebellum, ASIC1 staining was particularly strong in the molecular layer, and in both the molecular and granule cell layers, it was distributed diffusely, suggesting that its source is rather widespread (FIG. 5). In these layers, the most prevalent cell types are granule and Purkinje cells. Because both produce H⁺-evoked currents (Allen and Attwell, 2002; Escoubas et al., 2002; C. Askwith, unpublished observations), both probably contribute to the strong ASIC1 labeling.

The ASIC1 staining in the granule layer suggested that it may be distributed to granule cell dendrites, which are located there and receive afferent mossy fiber input. ASIC1 may also be present in granule cell axons, which project into the molecular layer where ASIC1 staining was strong (FIG. 5). However, because Purkinje cells are known to express large H⁺-gated currents, Purkinje cell dendrites may account for much of the ASIC1 protein in the molecular layer. A Purkinje cell-specific antibody (anti-calbindin D-28K) produced a similar diffuse pattern in the molecular layer (FIG. 5C). Purkinje cell axons traverse the white matter to form presynaptic terminals in the deep nuclei; however, ASIC1 staining in these areas was not greater than that in the −/− controls. The absence of detectable ASIC1 protein in Purkinje cell axons suggests that in these cells, it may be preferentially localized to dendrites.

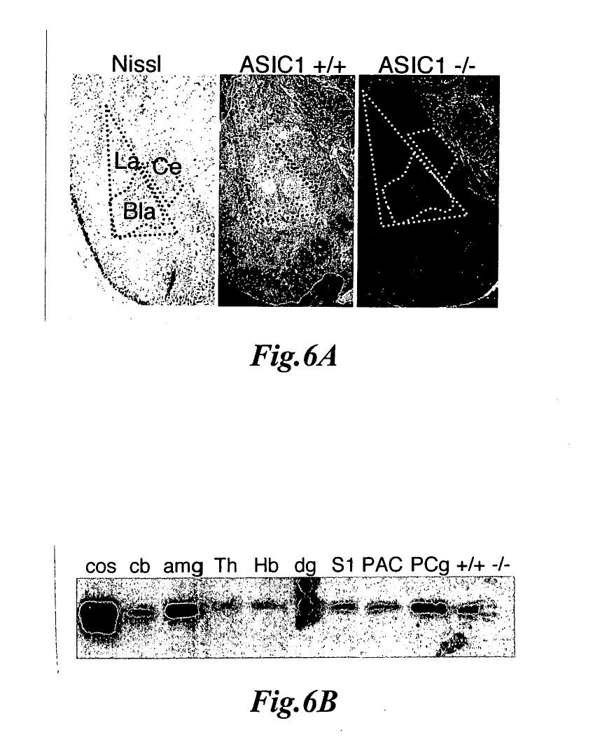

A general pattern that emerged in the study of ASIC1 localization in brain was a tendency for it to be enriched in areas receiving strong excitatory corticofugal input (cortical projections); examples include the cortex, striatum, nucleus accumbens, and dentate gyrus of the hippocampus. These structures are interconnected in a circuit referred to as the limbic corticostriatal loop (for review, see Cardinal et al., 2002). Components of this circuit are thought to contribute to the emotional importance of external stimuli and/or their expression. Another important component of this circuit is the amygdala complex, in which ASIC1 immunolabeling was intense, particularly in the lateral and basolateral nuclei (FIGS. 1A, 6A). The inventors obtained a similar result using Western blot to compare ASIC1 protein levels. ASIC1 was especially abundant in the amygdala and was present at higher levels than in the hippocampus or thalamus, for example (FIG. 6B).

These data are in contrast to those described recently by Alvarez de la Rosa et al. (2003), which suggested that ASIC1 protein was broadly distributed in neurons throughout the brain without a trend toward a particular brain region or cellular domain. One advantage of the present invention's experiments is that the inventors used ASIC1 knock-out mice as a control for specificity. Moreover, a multiple approaches, including immunohistochemistry, Western blotting, and measurement of H⁺-gated current density (see below), all suggest the ASIC1 protein is preferentially distributed to specific domains. These findings are also consistent with the inventor's earlier experiments in cultured neurons transfected with ASIC1, which showed a dendritic and synaptic pattern of ASIC1 localization (Wemmie et al., 2002).

ASIC1 is a Required Component of H⁺-Activated Channels in the Amygdala

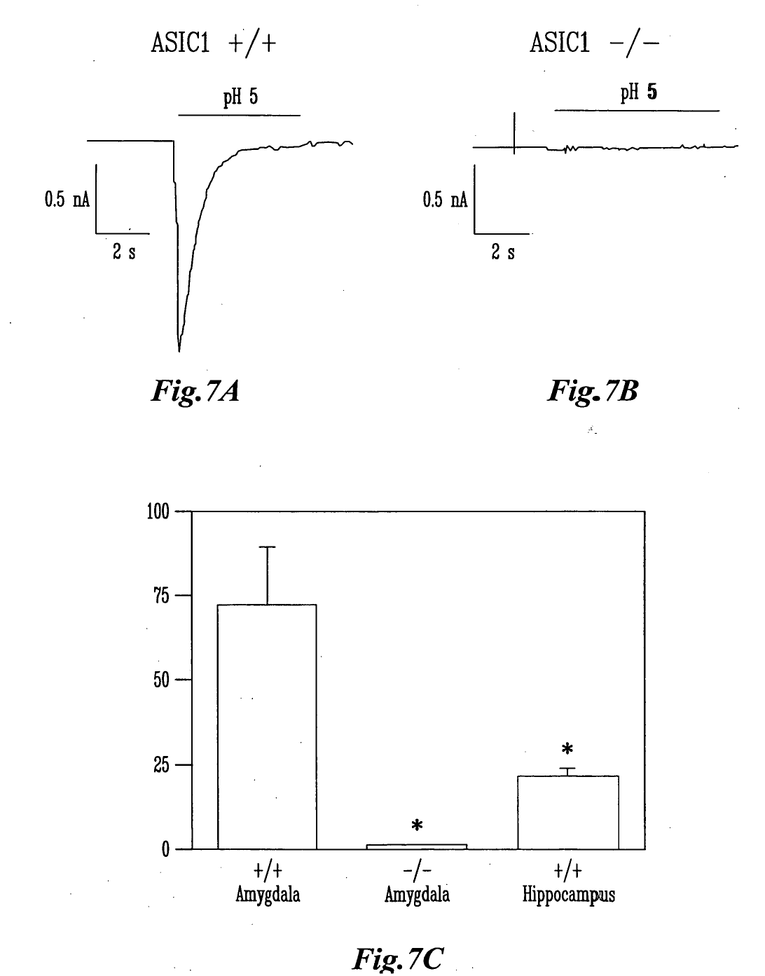

To explore the electrophysiological impact of ASIC1 expression in the amygdala, the present invention measured H⁺-gated currents in cultured amygdala neurons. Reducing extracellular pH to 5.0 evoked large transient currents in the majority of ASIC1 +/+ neurons (93% p n=27) (FIG. 7). In contrast, none of the amygdala neurons from ASIC −/− mice generated transient currents in response to pH 5 (n=29). These data indicate that ASIC1 makes a critical contribution to H⁺-gated current in these cells. It was also found that the mean current density of H⁺-gated currents was more than threefold greater in amygdala than in hippocampal neurons (FIG. 7). Thus compared with hippocampus, the amount of ASIC1 protein and the average number of functional ASIC channels are much greater in the amygdala.

ASIC1 and Amygdala-Dependent Behavior

Finding that ASIC1 protein was present in a number of structures in the limbic corticostriatal loop and that ASIC1 protein and H+-gated currents were abundant in the amygdala teaches that ASIC1 plays an important role in behaviors controlled by these structures (Cardinal et al., 2002). To test this hypothesis, the inventors examined the effect of ASIC1 disruption on performance in the elevated plus maze, a test of baseline fear. Both ASIC1 +/+ and −/− mice spent the majority of time in the closed arms (+/+=198±4 seconds; −/−=217±3 seconds; mean±SEM; p=0.21), suggesting that the two groups found the open arms similarly aversive. In addition, the number of open arm entries (+/+=12±1; −/−=12±1; mean±SEM; p=0.96), motor activity (time motionless in the corner of the closed arms, +/+=78±5 seconds; −/−=75±5 seconds; mean±SEM; p=0.88), and risk assessment (time scanning edge, +/+=16±0.5 seconds; −/−=14±0.7 seconds; mean±SEM; p=0.56) was similar for the two genotypes. Together, these data suggest that activity and baseline fear are normal in ASIC1 −/− mice.

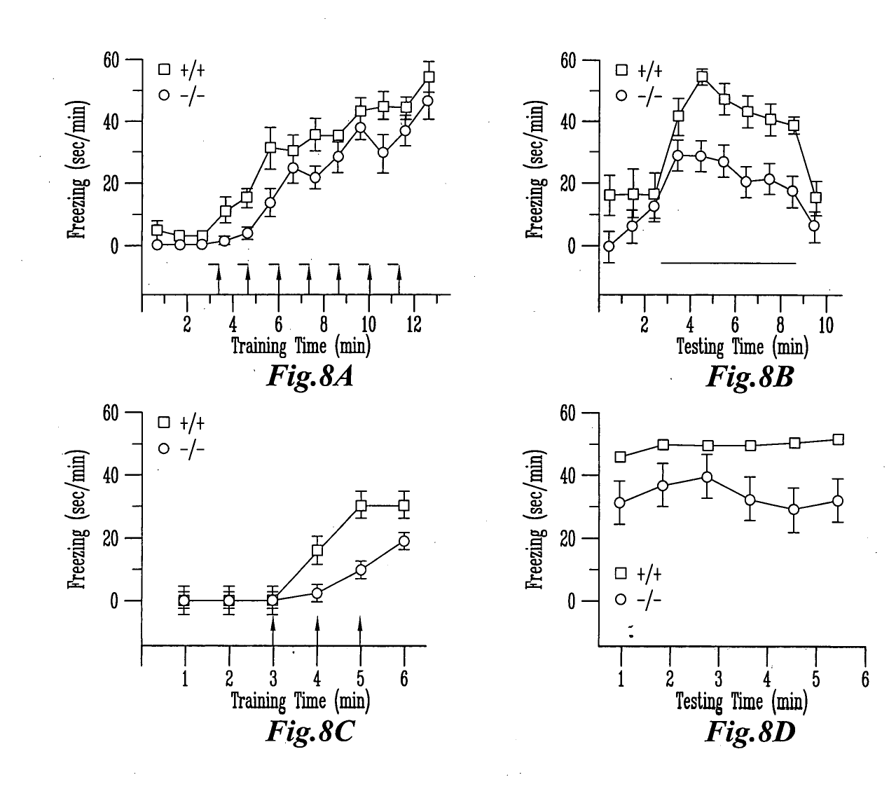

The amygdala is a key component of the circuitry for learned fear (Faneslow and LeDoux, 1999). The inventors' previous finding that ASIC1 disruption impaired synaptic plasticity and memory (Wemmie et al., 2002) raised the possibility that loss of ASIC1 would alter amygdala-dependent learning. The present invention tested cued fear conditioning by repeatedly presenting a tone and foot shock and measuring the percentage of time spent freezing during 1-minute intervals. With repeated stimuli, both the +/+ and −/− mice froze more, although the −/− mice lagged slightly behind. However, the robust freezing of −/− mice in the final minute of training (FIG. 8A) suggested that −/− animals were capable of expressing a strong fear response when trained extensively. The next day, the inventors tested the ability of the tone to induce freezing in the absence of a shock. A continuous tone was presented for 6 minutes. Animals of both genotypes responded with an increase in freezing, indicating the occurrence of auditory fear conditioning (FIG. 8B). However, the ASIC1-null mice spent significantly less time freezing than their wild-type littermates.

The presence of ASIC1 in the primary sensory cortex and sensory neurons raised the possibility that the −/− mice performed poorly because of a sensory deficit. However, after each shock without exception, both −/− and +/+ mice responded by jumping, vocalizing, or running. The average duration of the response (+/+, 1.7±0.2 seconds; −/−, 1.5±0.2 seconds; mean±SD; p=0.057), and the percentage of shocks eliciting a vocalization (+/+, 80.7±26.4%; −/−, 91.6±23.7%; p>0.2) was similar between the two groups. These results agree with the inventor's previous studies, which found that unconditioned responses to electrical shock during eyeblink conditioning were normal in ASIC1 −/− mice (Wemmie et al., 2002). In addition, at the behavioral level, it was found no differences in +/+ and −/= animals in mechanosensation, thermal sensation, or allodynia to skin or muscle stimulation (K. Sluka, personal communication; data not shown). Finally, both genotypes performed similarly on an accelerating Rotarod (Wemmie et al., 2002). Together, these data suggest that the observed differences in fear conditioning were not expected to have been the result of a sensory or motor deficit.

To test whether the fear conditioning deficit was restricted to cue, the mice were also conditioned to context. Again, the −/− mice acquired the freezing response more slowly on day 1 (FIG. 8C) and froze less on day 2, suggesting that the problem in fear conditioning is not restricted to auditory stimuli.

Having described the invention with reference to particular compositions, theories of effectiveness, and the like, it will be apparent to those skilled in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary. It is to be further understood that all citations to articles, etc., herein are hereby expressly incorporated in their entirety by reference.

What is claimed is:

1. A method of treatment for anxiety or an anxiety disorder comprising:
administering to a patient in need thereof a therapeutically effective amount of an acid sensing ion channel (ASIC) antagonist and a pharmaceutically acceptable carrier.

2. The method of claim 1 wherein the ASIC antagonist is an acid-sensing ion channel 1 (ASIC1) receptor antagonist.

3. The method according to claim 1 wherein said acid sensing ion channel (ASIC) antagonist and said pharmaceutically acceptable carrier is administered by a route selected from the group consisting of: orally, topically, sublingually, buccally, intranasally, rectally and intravenously.

4. The method of claim 1 wherein said anxiety or anxiety disorder is selected from the group consisting of:
generalized anxiety disorder, panic anxiety, obsessive compulsive disorder, social phobia, performance anxiety, post-traumatic stress disorder, acute stress reaction, adjustment disorders, hypochondriacal disorders, separation anxiety disorder, agoraphobia and specific phobias.

5. A method of treatment for drug addiction comprising:
administering to a patient in need thereof a therapeutically effective amount of an acid sensing ion channel (ASIC) antagonist and a pharmaceutically acceptable carrier.

6. The method of claim 5 wherein the ASIC antagonist is an acid-sensing ion channel 1 (ASIC1) receptor antagonist.

7. The method according to claim 5 wherein said acid sensing ion channel (ASIC) antagonist and said pharmaceutically acceptable carrier is administered by a route selected from the group consisting of: orally, topically, sublingually, buccally, intranasally, rectally and intravenously.

8. The method of claim 5 wherein said drug addiction is selected from the group consisting of:
addiction to alcohol, amphetamine, cocaine, heroin, inhalants, morphine, nicotine, opiates, psychoactive drugs, compulsive disorder, depression, headache, and drug or alcohol related withdrawal symptoms.

9. A method of treatment for fear conditioning comprising:
administering to a patient in need thereof a therapeutically effective amount of an acid sensing ion channel (ASIC) agonist and a pharmaceutically acceptable carrier.

10. The method of claim 9 wherein the ASIC agonist is an acid-sensing ion channel 1 (ASIC1) receptor agonist.

11. A method according to claim 9 wherein said acid sensing ion channel (ASIC) agonist and said pharmaceutically acceptable carrier is administered by a route selected from the group consisting of: orally, topically, sublingually, buccally, intranasally, rectally and intravenously.

12. A drug screening protocol method for identifying therapeutic agents for the treatment of anxiety, an anxiety disorder or drug addiction comprising:
providing an assay to determine modulation of the acid-sensing ion channel (ASIC) antagonist wherein the therapeutic agent deactivates the acid-sensing ion channel;
introducing to said assay a compound suspected of being an ASIC antagonist; and
determining whether ASIC modulation occurs wherein the therapeutic agent that modulates the level of expression of the acid-sensing ion channel is a candidate for the treatment of anxiety, anxiety disorders or drug addiction.

13. The method of claim 12 wherein the ASIC antagonist is an acid-sensing ion channel 1 (ASIC1) receptor antagonist.

14. The method of claim 12 wherein the assay to determine modulation of the acid-sensing ion channel (ASIC) is composed of cells that modulate the binding of an acid-sensing ion channel thereby inhibiting ASIC receptors.

15. A drug screening protocol method for identifying therapeutic agents for the treatment of fear conditioning comprising:
providing an assay to determine modulation of the acid-sensing ion channel (ASIC) agonist wherein the therapeutic agent activates the acid-sensing ion channel;
introducing to said assay a compound suspected of being an ASIC agonist; and
determining whether ASIC modulation occurs wherein the therapeutic agent that modulates the level of expression of the acid-sensing ion channel is a candidate for the treatment of fear conditioning.

16. The method of claim 15 wherein the ASIC antagonist is an acid-sensing ion channel 1 (ASIC1) receptor agonist.

17. The method of claim 15 wherein the assay to determine modulation of the acid-sensing ion channel (ASIC) is composed of cells that modulate the binding of an acid-sensing ion channel thereby activating ASIC receptors

18. A pharmaceutical composition for treatment of anxiety or an anxiety disorder comprising:
an acid-sensing ion channel 1 (ASIC1) receptor antagonist and a pharmaceutically acceptable carrier.

19. The pharmaceutical composition of claim 18 wherein said pharmaceutically acceptable carrier is selected from the group consisting of:
a carrier, diluent, excipient, wetting agent, buffering agent, suspending agent, lubricating agent, adjuvant, vehicle, delivery system, emulsifier, disintegrant, absorbent, preservative, and surfactant suitable for use in said pharmaceutical composition.

20. A pharmaceutical composition for treatment of drug addiction comprising:
an acid-sensing ion channel 1 (ASIC1) receptor antagonist and a pharmaceutically acceptable carrier.

21. The pharmaceutical composition of claim 20 wherein said pharmaceutically acceptable carrier is selected from the group consisting of:
a carrier, diluent, excipient, wetting agent, buffering agent, suspending agent, lubricating agent, adjuvant, vehicle, delivery system, emulsifier, disintegrant, absorbent, preservative, and surfactant suitable for use in said pharmaceutical composition

22. A pharmaceutical composition for treatment of fear conditioning comprising:
an acid-sensing ion channel 1 (ASIC1) receptor agonist and a pharmaceutically acceptable carrier.

23. The pharmaceutical composition of claim 22 wherein said pharmaceutically acceptable carrier is selected from the group consisting of:
a carrier, diluent, excipient, wetting agent, buffering agent, suspending agent, lubricating agent, adjuvant, vehicle, delivery system, emulsifier, disintegrant, absorbent, preservative, and surfactant suitable for use in said pharmaceutical composition

24. A method of treating a disease state associated with increased pH which comprises:
administering to a patient a therapeutically effective amount of an acid sensing ion channel (ASIC) antagonist and a pharmaceutically acceptable carrier.

25. The method of claim 24 wherein the disease state is an anxiety condition selected from the group consisting of:
generalized anxiety disorder, panic anxiety, obsessive compulsive disorder, social phobia, performance anxiety, post-traumatic stress disorder, acute stress reaction, adjustment disorders, hypochondriacal disorders, separation anxiety disorder, agoraphobia and specific phobias.

26. The method of claim 24 wherein the disease state is a drug addiction selected from the group consisting of:
addiction to alcohol, amphetamine, cocaine, heroin, inhalants, morphine, nicotine, opiates, psychoactive drugs, compulsive disorder, depression, headache, and drug or alcohol related withdrawal symptoms.

27. A method of preparing a pharmaceutical composition for treatment of anxiety or an anxiety disorder associated with a function of acid sensing ion channel 1 ASIC1, the method comprising:
(a) identifying a compound that modulates ASIC1;
(b) synthesizing the identified compound; and
(c) incorporating the compound into a pharmaceutical carrier.

28. A method of preparing a pharmaceutical composition for treatment of a drug addiction associated with a function of acid sensing ion channel 1 ASIC1, the method comprising:
(a) identifying a compound that modulates ASIC 1;
(b) synthesizing the identified compound; and
(c) incorporating the compound into a pharmaceutical carrier.

29. A method of preparing a pharmaceutical composition for treatment of fear conditioning associated with a function of acid sensing ion channel 1 ASIC1, the method comprising:
(a) identifying a compound that modulates ASIC1;
(b) synthesizing the identified compound; and
(c) incorporating the compound into a pharmaceutical carrier.

30. A method of treating a CNS disorder characterized by extracellular pH in the amygdala comprising:
inhibiting the acid-sending ion channel 1 (ASIC1) channel in a patient in need of such treatment.

31. The method of claim 30 wherein said CNS disorder is selected from the group consisting of:
generalized anxiety disorder, panic anxiety, obsessive compulsive disorder, social phobia, performance anxiety, post-traumatic stress disorder, acute stress reaction, adjustment disorders, hypochondriacal disorders, separation anxiety disorder, agoraphobia, specific phobias, addiction to alcohol, amphetamine, cocaine, heroin, inhalants, morphine, nicotine, opiates, psychoactive drugs, compulsive disorder, depression, headache, and drug or alcohol related withdrawal symptoms.
